SEQUENCE LISTING (2 sequences)

SEQ ID NO: 1 - DNA, 4533 bp, Homo sapiens
  CDS (23)..(2119)
gcgcgccggcggcacttccaacatggcggcggccggggcggcggtggcgcgcagcccgggaatcggagcgggacctgcgctgagagcccggcgctcgcccccgccgcgggccgcacggctgccgcggctgctagtgctgctagcggcggcggcggtggggccgggcgcgggcggtgcggcgcggctgtaccgcgcgggcgaggacgccgtgtgggtgctggacagcggcagcgtgcgcggggccaccgccaacagctcggccgcgtggctcgtgcagttctactcgtcgtggtgtggccactgcatcggctacgcgcccacttggcgggccctggctggggatgtgcgagactgggccagtgccattcgcgtcgcagctctggactgcatggaagagaagaaccaggccgtgtgccatgactacgacatccacttctaccccaccttccggtattttaaagcatttacaaaggagtttacaactggagaaaattttaaaggacctgaccgagagctgcgaacagtcagacagacgatgattgacttcctgcagaaccacacggaaggaagccggccccctgcctgcccgcgcctagaccccattcagcccagtgatgttctttcccttcttgacaaccgtggcagccattacgtggctattgtctttgaaagcaacagctcctaccttggacgggaggtgatcttagacctgatcccgtatgaaagcatcgtggtgacccgagcactggacggggacaaagcatttctggagaaacttggtgtttcttcagtcccttcgtgttacctgatctacccaaatgggtcgcatggattgattaacgtcgtgaagcctctgcgggccttcttttcgtcttatttgaagtcattgccggatgtgaggaaaaaatcgcttcccttgcctgaaaagccacacaaagaagaaaattcagaaatcgtggtttggagagaatttgacaagtcgaagctgtacacggtggacctggagtcagggctacactacctcctgcgggtggagctggcagcccacaagtccctggccggagcagagctgaagacgctcaaggactttgtgactgtcttggccaagctgttccctggacggccgccagtcaagaagctgttggagatgctgcaggagtggctggccagccttcccctggacaggatcccctacaacgccgtgcttgacctggtcaacaacaagatgcggatttctggaatattccttactaatcacataaagtgggttggatgtcaaggaagccgatctgagttgaggggttacccgtgttctctctggaaactgttccacactttgactgttgaagcctcgacccacccagatgcactggttggcacaggctttgaagacgacccccaggctgtgctgcagacaatgaggaggtacgttcacaccttctttgggtgtaaggaatgtggtgagcactttgaggaaatggctaaagaatccatggactcggtgaaaaccccagaccaagccatcctctggctgtggaagaagcataatatggtgaacggccgcctggcaggccatctgagtgaggatccccggtttccaaagcttcagtggcccactccggacctctgcccagcctgccatgaggaaattaagggcctggccagctgggatgaaggccacgtgctcacattcttgaagcagcactatggccgcgacaacctcttagacacgtattccgcagaccagggggattccagtgaaggaggaaccctggccaggggtgaggaagaggagaaaagactcactcccccagaggtgtcccatggagaccgagacacccagagcgtccgtccacctggtgcactgggccccaggcctgcccttccagagagcttgcatcacagcttggacgggaaactccagagtctggatgggcccggggcccacaaggaggtgggcggggccgcacccttcctcggggttgacttctccagcctggacatgagtctctgtgtcgtgctgtacgtggcttcatccctgttcctcatggtgatgtacttcttcttccgggtgaggtccaggcggtggaaggtcaagcaccaccacccggccgtgtgagtgcccgggtgctgccagccacggcggaagctcccttggaggcagccctgccccgtgccacctgcagctttaatatttatgatcagggattttataaacatgcgggcctggtttcacatcggatggcaccttttggcttcaaagtcctggttttacaaacgctcttctaacaagggaagaacacggggtaattttgtggggatatttgcattcctggcgtactcaagtctgttcatgctccttttgcaggtcttacagcaaaaagacttctgtatttttactcttctagatgtgaaaagagggtgcagagtccaggccagatagtcttccccacacactttcatctcgtttcctccactccgccccatcctgcagggccttgtttttgtatttggagaacctcgcccatcccccccgcggctcctggctccccccccccccaccttggccctccctgcaccctccactcccctcgctccccctccccccgctccccccacgccccctgctccaggctgccaagtgttttcctttagccgggcggggacagacagagccggaagcgcagtcggcctctgcagcccctccaagcaagtgctccagggactatcctgtgtttgtagctgcttccctagggcaggttcctgagggctctccttgttcctccgggtgttcgacaccagacgtggggatttcaacaggggaggagccaaggaattctgtggctgtgctgcgtttcagaaaataacccccagaggccttgggctgtggacctgggggttggaaggatgggggctcatttaaccctcagaggcagcgcctttgtctgtctatctggtgacaagagagagacaagtaaatgggggccgttgggacggcgggtgcctggagggcagctctgggctcagcgggcagtgcttagagcacaggcccctctgttgggggatggggaggagagcagtctgcccttgggagcgtaggccccagggagacttctaaagccccccctgtcgtctgctcttcacccagcaccacagaggcacctgctgcacacacaagcatctcactcggcccacggagggggccaggcttcctttgcctgaagctgttttgggaagggtctccacacaggcactgatctcccaagctttggtcatgatgtcttttaccatttgataattttaaacattgtttttaaacccaaaacatttagtggtccgttgcctctgaagatgtaaacaaacaaatacactatttctgggaacatttatattgagattctttgtggctattggtgtgtctcacaggcaaaatttgatttggctaaaataggctcagatgtatttgtgtgcccgtgtgtgtgtgtgtgtgtgtgtgtgtgtgtgtgtatgagagagagagagactttgacggttgtagatattttttccgctttgcctactttatgttgtataatcatgtgtttactaacaagttgatgacatggatgtattcataagaccatgtaatattgatgtgattgttgtcgcttgagaaaaaaaggcaacagctgattctttcaacaactgtcacagaatggctgggctgagaacgctgcccagggccctgcagctggcgggagaggtgtctggtgggagcggtgtctggtgcgtcagcctgctgcttcgtgtctcactcgagagttgcttctggtttcacactttttaacccctctgtgctttagcagccgtgaccttgccttcattgcttcatccagtgcaggcctgggttattgaagacaccaaagtgtttctcttccagtttgaaaaccaggcaggtttacacgtgggtttcagtgtatttgcctttgaacccttcaaactaaactttagccttttggctggtgttgaatgtctttagctggggtgacctggagtgacctgggacggtgttgtggtttaccgtctccagcttcagcctttccagaaacccttgtggagggcagtgttggctgcaggtttcatcatattgcagtttgaggtcaccactatttggggaacaagcttgcgtcctgctgaggggcaggattttcagcagagcgtcggtggggctgggccgtcgtggaggtggccccaggagtcatatggccatactcagacacaccttgtgtggcctgctcagagctggatgccacctttaggcaatgtttagagtctattttctaaagttttaagtattttaagaggtattgagactaatgaatataatagttcagtaatttaaatgcttatttattttcagtggaaggatttttattaaaaagaagctaattgacatggaaatgtcagtgaaatttcttacctgcaaggaaagtgaacattttgtatttaagtaaactataatgtgcacattttaataaagaaatctgacatttg

SEQ ID NO: 2 - DNA, 44 bp, Artificial Sequence
  Description of artificial sequence  Synthetic oligonucleotide primer
gactagttctagatcgcgagcggccgcccttttttttttttttt

(19) United States
(12) Patent Application Publication
(10) Pub. No.: US 2006/0160082 A1
(43) Pub. Date: Jul. 20, 2006

(54) Novel genes and proteins encoded thereby

(75) Inventors: Osamu Ohara, Kisarazu-shi (JP); Takahiro Nagase, Kisarazu-shi (JP); Reiko Kikuno, Kisarazu-shi (JP)

Correspondence Address: BIRCH STEWART KOLASCH & BIRCH, PO BOX 747, FALLS CHURCH, VA, 22040-0747, US

(73) Assignees: Kazusa Dna Research Institute Foundation, Kisarazu-Shi, Chiba (JP); ProteinExpress Co. Ltd, Choshi-Shi, Chiba (JP)

(21) Appl. No.: 10/530,880

(22) PCT Filed: Oct. 16, 2003
(86) PCT No.: PCT/JP03/13276

§ 371 Date: Oct. 27, 2005

(30) Foreign Application Priority Data
Oct. 21, 2002 (JP) 2002305318

(51) Int. Cl. C12Q 1/68; C07H 21/02; C12P 21/06; C07K 14/705; C07K 16/28

(52) U.S. Cl. 435/6; 435/69.1; 435/320.1; 435/325; 530/350; 530/388.22; 536/23.1

(57) ABSTRACT

A novel DNA comprising a protein-encoding region is directly cloned from cDNA library originating from human adult whole brain and human fetal whole brain, the base sequence thereof is determined, and the functions thereof are identified. A DNA comprising a base sequence encoding the following polypeptide (a) or (b): (a) a polypeptide consisting of an amino acid sequence which is identical or substantially identical with the amino acid sequence represented by SEQ ID No.1, (b) a polypeptide consisting of an amino acid sequence represented by SEQ ID No.1 in which part of amino acids are deleted, substituted or added, and having substantially the same biological activity as the function of the polypeptide (a), recombinant polypeptide encoded by the above DNAs, and protein comprising the polypeptide.

### FIELD OF THE INVENTION

The present invention is related to a novel DNA and a gene comprising the DNA, a recombinant polypeptide encoded by the DNA and a novel recombinant protein comprising the polypeptide. More particularly, it is related to a novel protein that is supposed to belong to Quiescin Q6 family and a gene encoding thereof.

### BACKGROUND OF THE INVENTION

A grand scale sequencing in the Human Genome Project has been producing and analyzing a lot of information on the nucleotide sequences of human genome every day.

A final goal of the project is not only to determine the whole genomic nucleotide sequences, but also to reveal and understand various human life phenomena based on the information about their structure, i.e., DNA sequence information.

[Non-Patent Document 1]

Donald L. Coppock, et al., Genomics 54, “The Quiescin Q6 Gene (QSCN 6) is a Fusion of Two Ancient Gene Families: Thioredoxin and ERV1”, 1998, p.460-468

[Non-Patent Document 2]

Beatrice Beayoun, et al., The Journal of Biological Chemistry Vol.276, No.17, “Rat Seminal Vesicle FAD-dependent Sulfhydryl Oxidase”, 2001, p.13830-13837.

Regions encoding proteins occupy only a small part of the human genome. Although the coding region may recently be predicted by utilizing techniques in information technology such as neural network and hidden markov model, their predictive accuracy is not yet enough.

### SUMMARY OF THE INVENTION

The present inventors have succeeded in directly cloning a novel DNA comprising a region encoding a protein from cDNA library derived from human adult whole brain and human fetal whole brain, and in determining its nucleotide sequence, and have completed the present invention.

A first aspect of the present invention relates to a DNA comprising a nucleotide sequence encoding the following polypeptide (a) or (b):
(a) a polypeptide consisting of an amino acid sequence which is identical or substantially identical with an amino acid sequence represented by SEQ ID No.1,
(b) a polypeptide consisting of an amino acid sequence represented by SEQ ID No.1 in which part of amino acids are deleted, substituted or added, and having substantially the same biological activity (function) as the function of the polypeptide (a).

One example of the above DNA is that comprising a base sequence represented by SEQ ID No.1.

A second aspect of the present invention relates to a DNA hybridizing with a DNA having a base sequence complementary to the DNA of the first aspect of the present invention under stringent conditions, and having substantially the same biological activity as the function of the above polypeptide (a).

The DNAs of the first and second aspects will be also referred to as “the present DNA” in the present specification. The present invention is also related to an antisense DNA having a base sequence that is substantially complementary to the present DNA.

A third aspect of the present invention relates to a gene construct comprising the present DNA. The term “gene construct” in the present specification means any gene that is artificially manipulated. The gene construction includes, for example, a vector comprising the present DNA or antisense DNA, and an expression vector comprising the present DNA.

A forth aspect of the present invention relates to A polypeptide (a) or (b):
(a) a polypeptide consisting of an amino acid sequence which is identical or substantially identical with the amino acid sequence represented by SEQ ID No.1,
(b) a polypeptide consisting of an amino acid sequence represented by SEQ ID No.1 in which part of amino acids are deleted, substituted or added, and having substantially the same biological activity as the function of the above polypeptide (a).

A fifth aspect of the present invention relates to a recombinant polypeptide encoded by the gene construct of the third aspect of the present invention.

The above polypeptides are also referred to as “the present polypeptide” in the present specification. The term “polypeptide” in this specification means a “polymer of amino acids having any molecular weight.” The present invention may contain a recombinant protein comprising the present polypeptide. As there is no limitation in the molecular weight with respect to the polypeptide of the present invention, the present polypeptide may also contain the recombinant protein.

A sixth aspect of the present invention relates to an antibody against the present polypeptide.

A seventh aspect of the present invention relates to a DNA tip on which the present DNAs are arrayed.

A eighth aspect of the present invention relates to a polypeptide tip on which the present polypeptides are arrayed.

A ninth aspect of the present invention relates to an antibody tip on which the antibodies of the sixth aspect of the present invention are arrayed.

A tenth aspect of the present invention relates to an antisense oligonucleotide to the present DNA.

### BEST MODE FOR CARRYING OUT THE INVENTION

The present DNA is isolated as cDNA fragment from a cDNA library prepared by the present inventors by using as starting materials mRNAs of human adult whole brain and human fetal whole brain, which are commercially available from Clontech, and identified with determination of its nucleotide sequence.

Thus, clones are randomly isolated from the library derived from human adult whole brain and human fetal whole brain, which is prepared in accordance with Ohara et al., DNA Research Vol.4, 53-59 (1997).

Homology search is done on databases of known genes with the use of the nucleotide sequences at both ends thus obtained clones as a query to find new clones. The 5′ and 3′ terminal sequences of the new clones are aligned with the human genome. When an unknown gene with a long base sequence is found, a whole length analysis is done for the unknown gene.

Paying much attention not to make any artificial errors in short fragments or determined sequences, the whole region of human genes comprising the present DNA may be prepared also by using PCR methods such as RACE.

The present invention further relates to a recombinant vector comprising the present DNA or the gene construct comprising the present DNA, to a transformant harboring the recombinant vector, to a method for the production of the present polypeptide, the recombinant protein comprising the polypeptide or salts thereof comprising culturing the transformant, and producing, accumulating and recovering the present polypeptide or the recombinant protein comprising the polypeptide, and to the present polypeptide, the recombinant protein comprising the polypeptide or salts thereof thus obtained.

The present invention still further relates to a pharmaceutical composition comprising the present DNA or gene construct, to a polynucleotide (DNA) encoding the present polypeptide or partial polypeptide thereof or the recombinant protein comprising the polypeptide, to the antisense oligonucleotide to the present DNA, to a pharmaceutical composition comprising the above polynucleotide or the antisense nucleotide, and to pharmaceutical composition comprising the present polypeptide or partial polypeptide thereof or the recombinant protein comprising the polypeptide.

The present invention also relates to the DNA tip, peptide tip and antibody tip that are prepared by arraying the present DNAs, the present polypeptides and the antibodies against the present polypeptides, respectively.

Still the present invention relates to a method for screening of a compound which specifically interact with the present polypeptide or partial polypeptide thereof, the recombinant protein comprising the polypeptide, salts thereof or antibodies against them by using these materials, a screening kit, and a compound identified by the screening method.

The present DNA may be any DNA as long as it comprises the base sequence encoding the present polypeptide, including a cDNA identified and isolated from cDNA libraries derived from human brain and other tissues or cells such as heart, lung, liver, spleen, kidney and testis, and a synthetic DNA.

A vector, which is used in the preparation of the libraries, includes bacteriophage, plasmid, cosmido and phagemid. The cDNA may be also amplified by means of Reverse Transcription coupled Polymerase Chain Reaction (RT-PCR) with the use of a total RNA or mRNA fraction prepared from the above tissues or cells.

The antisense oligonucleotide (DNA) to the present DNA may includes any antisense DNA having the base sequence that is substantially complementary to the DNA encoding the present polypeptide or the partial polypeptide thereof, and having a function of inhibiting the expression of the present DNA.

The “base sequence that is substantially complementary to the present DNA” means, for example, a base sequence having homology of about 90% or more, preferably about 95% or more, more preferably about 100% to the whole or partial base sequence complementary to the present DNA. Any nucleic acid sequence having the function similar to that of the above antisense DNA such as a modified RNA or DNA may be included in the antisense oligonucleotide of the present invention. These antisense oligonucleeotide may be produced by a known DNA synthesizer or the like.

The “amino acid sequence which is substantially identical with an amino acid sequence represented by SEQ ID No.1” means an amino acid sequence having homology on an average of about 70% or more, preferably about 80% or more, more preferably about 90% or more, further more preferably about 95% or more to the whole amino acid sequence represented by SEQ ID No.1.

Thus, a polypeptide consisting of the amino acid sequence which is substantially identical with the amino acid sequence represented by SEQ ID No.1 includes a polypeptide having the above homology to the amino acid sequence represented by SEQ ID No.1 and having substantially the same biological activity (or function) as the function of a polypeptide consisting of the above amino acid sequence. The term “substantially the same” means the activities or functions of the both substances are the same with each other in quality or property.

The present polypeptide includes a polypeptide consisting of the amino acid sequence represented by SEQ ID No.1 in which part of amino acids (preferably 1˜20, more preferably 1˜10, further more preferably a few amino acids) are deleted, substituted or added, and having substantially the same biological activity (or function) as the function of the polypeptide consisting of the amino acid sequence represented by SEQ ID No.1.

The polypeptide consisting of the amino acid sequence which is substantially identical with the amino acid sequence represented by SEQ ID No.1, or the polypeptide consisting of the amino acid sequence represented by SEQ ID No.1 in which part of amino acids are deleted, substituted or added may be easily prepared by well known methods such as site-specific mutation, genetic homologous recombination, primer extension method and PCR, or any optional combinations thereof.

In order for the polypeptide or protein to have substantially the same biological activity, it is possible to make a substitution among amino acids belonging to the same group (polar, non-polar, hydrophobic, hydrophilic, positive-charged, negative-charged, or aromatic amino acid group) in the amino acids that constitute the present polypeptide. Alternatively, it is desirable to keep amino acids which are included in a functional domain.

The present DNA includes the DNA comprising the base sequence encoding the polypeptide represented by SEQ ID No.1, and the DNA hybridizing with the DNA complementary to the DNA of the first aspect under stringent conditions and having substantially the same biological activity as the function of the polypeptide consisting of the amino acid sequence which is identical or substantially identical with the amino acid sequence represented by SEQ ID No.1.

The DNA that may hybridizes under the above stringent conditions includes a DNA having homology on an average of about 80% or more, preferably about 90% or more, more preferably about 95% or more to the whole base sequence of the above DNA

Hybridization may be performed in accordance with a method described in, for example, Current protocols in molecular biology (edited by Frederick M. Ausubel et al., 1987). If a commercial library is used it may be done according to a method described in instructions attached thereto.

The phrase “stringent conditions” in this specification means conditions under which Southern blot hybridization is carried out in an aqueous solution containing 1 mM Na EDTA, 0.5M Na₂HPO₄ (pH 7.2) and 7% SDS at 65° C., followed by the washing of a membrane with an aqueous solution containing 1 mM Na EDTA, 40 mM Na₂HPO₄ (pH 7.2) and 1% SDS at 65° C. The same stringency may be realized by using other conditions.

The present DNA may be cloned by preparing a synthetic DNA primer with an appropriate nucleotide sequence such as a part of the polypeptide of the present invention, and amplifying it with an appropriate library by means of PCR. The present DNA may be further selected from DNAs integrated into appropriate vectors by means of hybridization with a DNA fragment or synthetic DNA encoding the whole region or part of the present polypeptide.

Hybridization may be performed in accordance with a method described in, for example, Current protocols in molecular biology (edited by Frederick M. Ausubel et al., 1987). If a commercial library is used it may be done according to a method described in instructions attached thereto.

The present DNA thus cloned may be directly used, or optionally digested with a restriction enzyme or tagged with a linker for use. The present DNA may have a translation initiation codon “ATG” at its 5′-end, and a translation termination codon, “TAA”, “TGA” or “TAG” at its 3′ end. These codons may be also added by using an appropriate synthetic DNA adapter.

The expression vector may be constructed by any known method in the art. For example, it is made by (1) excising a DNA fragment containing the present DNA or the gene comprising the DNA, and (2) ligating the DNA fragment downstream of a promoter in the expression vector.

Vectors to be used in the present invention include those derived from Escherichia coli such as pBR322, pBR325, pUC18, pUC118; those derived from Bacillus subtilis such as pUB110, pTP5 and pC194; those derived from yeast such as pSH19 and pSH15; bacteriophage such as λphage; animal viruses such as retorovirus, vaccinia virus and baculovirus.

Promoters to be used in the present invention may be any promoters suitable for a host cell which is used in the expression of the gene, including, for example, trp promoter, lac promoter, recA promoter, λPL promoter and 1 pp promoter for E. coli; SPO1 promoter, SPO2 promoter and penP promoter for Bacillus subtilis; PHO5 promoter, PGK promoter, GAP promoter and ADH promoter for yeast; and SRα promoter, SV40 promoter, LTR promoter, CMV promoter and HSV-TK promoter for animal cells.

Other elements known in the art such as an enhancer, a splicing signal, a polyadenylation signal, a selection marker and SV40 replication origin may be added to the expression vectors. The protein encoded by the present DNA may be optionally expressed as a fused protein with other proteins such as glutathione-S-transferase and protein A. The fused protein may be cleaved by an appropriate protease and separated into each protein.

The host cell used in the present invention includes Escherichia, Bacillus, yeast, insect cells, and animal cells.

The examples of Escherichia include E. coli K-12•DH1 (Proc. Natl. Acad. Sci., USA, vol.60 160 (1968)), JM103 (Nucleic Acids Research, vol.9, 309 (1981)), JA221 (Journal of Molecular Biology, vol.120, 517 (1978)) and HB101 (Journal of Molecular Biology, vol.41, 459 (1969)).

The examples of Bacillus include Bacillus subtilis MI114 (Gene vol.24, 255 (1983)), and 207-21(Journal of Molecular Biology, vol.95, 87 (1984)).

The examples of yeast include Saccaromyces cerevisiae AH22, AH22R-, NA87-11A, DKD-5D, and 20B-12; Schizosaccaromyces pombe NCYC1913, NCYC2036; and Saccaromyces picjia pastoris.

The examples of animal cells include simian cell COS-7, Vero, Chinese hamster cell CHO (“CHO cell”), dhfr gene-defective CHO cell, mouse L cell, mouse AtT-20 cell, mouse myeloma cell, rat GH3 cell and human FL cell.

The transfomation of these cells may be carried out in accordance with a method known in the art such as those described in the following articles:

Proc. Natl. Acad. Sci., USA vol.69, 2110 (1972); Gene, vol.17, 107(1982), Molecular & General Genetics, vol.168, 111 (1979); Methods in Enzymology, vol. 194, 182-187 (1991); Proc. Natl. Acad. Sci., USA vol.75, 1929 (1978); Cell Engineering, additional volume 8, “New Cell Engineering experimental protocols, 263-267 (published by Shu-junn Co.); and Virology vol.52 456 (1973).

The transformant thus transformed with the expression vector comprising the present DNA or the gene comprising thereof may be cultured according to a method known in the art.

Escherichia host cells may be normally cultured at about 15˜43° C. for about 3˜24 hours with aeration and stirring, if necessary. Bacillus host cells may be normally cultured at about 30˜40° C. for about 6˜24 hours with aeration and stirring, if necessary.

Yeast host cells may be normally cultured in a culture medium with pH about 5˜8 at about 20˜35° C. for about 24f˜72 hours with aeration and stirring, if necessary.

Animal host cells may be normally cultured in a culture medium with pH about 6˜8 at about 30˜40° C. for about 15˜60 hours with aeration and stirring, if necessary.

The polypeptide or protein according to the present invention may be isolated and purified from the above culture as follows. After the completion of culturing, bacteria or cells are collected by a known method, suspended in an appropriate buffer solution, and destroyed by means of ultrasonic, lysozyme and/or freezing and thawing treatment, followed by centrifugation or filtration to give a crude protein extract. The buffer solution may contain a protein-denaturing agent such as urea and guanidine hydrochloride, or a surfactant such as TritonX-100™. If the protein is secreted into the culture medium, the bacteria or cells are separated from its supernatant by a known method after the completion of culturing, and the resulting supernatant is collected. The protein thus obtained and contained in the culture supernatant or extract may be purified by an appropriate combination of known separation and purification methods.

The present polypeptide or protein thus obtained may be converted into their salt form, which may be converted into its free from vice versa or into other salt forms according to a known method. The protein produced by the transformant may be treated with an appropriate protein-modifying enzyme such as trypsin or chymotrypsin in order to optionally add modification to it or to partially remove polypeptide from it before or after purification.

The presence of present polypeptide or protein or salt thereof may be determined by various binding assay methods or enzyme immunoassay using a specific antibody.

The C-terminus of the polypeptide of the present invention is normally a carboxyl group (—COOH) or a carboxylate (—COO—), but the C terminus may be an amide (—CONH₂) or ester (—COOR). Examples of R in ester that are used herein include a C1-6 alkyl group, such as methyl, ethyl, n-propyl, isopropyl or n-butyl; a C3-8 cycloalkyl group, such as cyclopentyl or cyclohexyl; a C6-12 aryl group, such as phenyl or α-naphthyl; a phenyl-C1-2 alkyl group, such as benzyl or phenethyl; and a C7-14 aralkyl group, such as an α-naphthyl-1-2 alkyl group, e.g., α-naphthyl methyl. Further, pivaloyl-oxymethyl ester generally used as oral administration may also be used.

When the polypeptide of the present invention has a carboxyl group (or carboxylate) other than at the C-terminus, the polypeptide of the present invention encompasses such a polypeptide wherein the carboxyl group is amidated or esterified. An example of the ester that is used in this case is the above-mentioned ester at the C-terminus. Moreover the polypeptide of the present invention also encompasses a polypeptide wherein an amino group of a methionine residue at the N-terminus is protected with a protecting group (for example, a C1-6 acyl group, such as a formyl group or an acetyl group); a polypeptide wherein a glutamic acid residue at the N-teminus which is generated by in vivo cleavage is pyroglutamated; a polypeptide wherein OH, COOH, NH₂, SH and the like on the side chain of intramolecular amino acids are protected with appropriate protecting groups ( for example, a C1-6 acyl group, such as a formyl group and an acetyl group); or a complex protein, such as a so-called glycoprotein formed by the binding of sugar chains to a polypeptide, or the like.

A partial polypeptide of the polypeptide of the present invention may be any partial peptide of the above-mentioned polypeptide of the present invention and has substantially the same activities. For example, a polypeptide that is used herein comprises a sequence of at least 10 or more, preferably 50 or more, further preferably 70 or more, further more preferably 100 or more, and most preferably 200 or more amino acids of the amino acid sequence of the polypeptide of the present invention, and, for example, has substantially the same biological activity with the function of the polypeptide of the present invention. An example of a preferable partial polypeptide of the present invention contains each functional domain. Further, the partial peptide of the present invention normally has a carboxyl group (—COOH) or a carboxylate (—COO—) at the C-terminus, and it may also have an amide (—CONH₂—) or an ester (—COOR) at the C-terminus like the above polypeptide of the present invention may have. Further, examples of the partial peptide of the present invention, similar to the polypeptide of the present invention described above, include a peptide wherein an amino group of a methionine residue at the N-terminus is protected with a protecting group; a peptide wherein a glutamyl residue at the N-terminus which is generated by in vivo cleavage is pyroglutamated; a peptide wherein a substituent on the side chain of intramolecular amino acids is protected with an appropriate protecting group; a complex peptide, such as a so-called glycopeptide formed by the binding of sugar chain to a peptide, or the like. The partial peptide of the present invention may be used as, for example, a reagent, reference or standard materials for experiments, or an immunogen or a portion thereof.

Particularly preferred salts of the polypeptide of the present invention or the partial peptide are physiologically acceptable acid-added salts. Examples of such salts that are used herein include a salt formed with an inorganic acid (for example, hydrochloric acid, phosphoric acid, hydrobromic acid and sulfuric acid), and a salt formed with an organic acid (for example, acetic acid, formic acid, propionic acid, fumaric acid, maleic acid, succinic acid, tartaric acid, citric acid, malic acid, oxalic acid, benzoic acid, methane sulfonic acid and benzenesulfonic acid).

The polypeptide of the present invention, the partial peptide thereof or salts thereof, or amides thereof can be prepared by a chemical synthesis method known in the art.

For example, amino acids whose α-amino groups and side chain functional groups are appropriately protected are condensed on resin (which is commercially available resin for protein synthesis) in accordance with the sequence of a target polypeptide, according to various condensation methods known in the art. Various protecting groups are then removed simultaneously with cleavage of the polypeptide from the resin at the end of reaction. Further, reaction for forming an intramolecular disulfide linkage is conducted in a highly diluted solution, thereby obtaining a target polypeptide, the partial peptide thereof or amides thereof. Examples of activation reagents that may be used to condense the above protected amino acids include those that may be used for polypeptide synthesis and are represented by carbodiimides, such as DCC, N,N′-diisopropylcarbodiimide and N-ethyl-N′-(3-dimethylaminopropyl) carbodiimide. For activation by such reagents, both protected amino acids and a racemization-suppressing additive (for example, HOBt or HOOBt) are directly added to the resin; or protected amino acids may be activated in advance as corresponding acid anhydride, HOBt ester or HOOBt ester, and then added to the resin.

Solvents used for the activation of protected amino acids and condensation with resin may be appropriately selected from solvents known in the art as applicable to polypeptide condensation reaction, such as acid amides, halogenated hydrocarbons, alcohols, sulfoxides and ethers. A reaction temperature is appropriately selected from a known range that may be used for reaction of polypeptide linkage formation. Activated amino acid derivatives are normally used in an amount of 1.5 to 4-fold excess. When condensation is found to be insufficient as a result of a test using ninhydrin reaction, condensation reaction without eliminating protecting groups will be repeated for sufficient condensation. When condensation is found to be still insufficient, unreacted amino acids are acetylated using acetic anhydride or acetylimidazole so as not to affect the subsequent reaction.

Protecting groups normally employed in the art may be used for each of amino groups, carboxyl groups and serine hydroxyl groups of raw materials.

The protection of functional groups that should not involve the reaction of raw materials, protecting groups, and the elimination of the protecting groups, and the activation of functional groups that involve reaction and the like may be appropriately selected from known groups or performed by known measures.

The partial peptide of the present invention or a salt thereof may be produced according to a peptide synthesis method known in the art, or by cleaving the polypeptide or the present invention with an appropriate peptidase. For example, the peptide synthesis method may be either a solid-phase synthesis method or a liquid phase synthesis method. Example of the known condensation method and the method of elimination of protecting groups are described in Nobuo IZUMIYA et al., Basics and Experiment for Peptide Synthesis, Maruzen (1975); Haruaki YAJIMA and Shunpei SAKAKIBARA, Experiment Course for Biochemistry 1, Protein Chemistry IV, 205 (1977); and Development of Pharmaceutical Preparation, vol. 14, Peptide Synthesis, under the editorship of Haruaki YAJIMA, Hirokawa Publishing Co.

After reaction, the partial peptide of the present invention may be purified and isolated by an appropriate combination of known methods, such as solvent extraction, distillation, column chromatography, liquid chromatography, and re-crystallization. When the partial peptide obtained by the above methods is a free one, it can be converted to an appropriate salt by a known method. Conversely, when the peptide is obtained as a salt, it can be converted to a free one by a known method.

The antibody against the polypeptide of the present invention, the partial peptide thereof or salts thereof may be either a polyclonal or a monoclonal antibody, so far as it can recognize these substances. The antibody against the polypeptide of the present invention, the partial peptide thereof or salts thereof may be produced using as an antigen the polypeptide of the present invention or the partial peptide thereof according to a known method for producing antibodies or anti-serum.

The antibody of the present invention may be used to detect the polypeptide of the present invention and the like which are present in a specimen, such as body fluid, tissues or the like. In addition, the antibody may be used for preparing an antibody column to be used for purifying these substances; detecting the polypeptide of the present invention in each fraction upon purification; analyzing the behavior of the polypeptide of the present invention within the cells of a specimen; and the like.

The use of the DNA, the polypeptide and the antibody of the present invention will be further described below.

Abnormalities (of the gene) in DNA or mRNA encoding the polypeptide of the present invention or the partial peptide thereof may be detected using as a probe the DNA of the present invention, the antisense DNA of the DNA of the present invention, or a gene construct comprising these DNAs.

The DNA, the antisense DNA or the gene construct of the present invention are useful as a genetic diagnostic agent for, for example, damages, mutation or hypoexpression in the DNA or mRNA, and an increase or hyperexpression of the DNA or mRNA. The above gene diagnosis using the DNA of the present invention may be performed by, for example, a known northern hybridization or a PCR-SSCP method (Genomics, 5:874-879 (1989), Proc. Natl. Acad. Sci. USA, 86:2766-2770(1989)).

Moreover, for patients whose function does not works in vivo because of abnormalities or deletion in the DNA or the gene of the present invention, or because the expression amount of the DNA or the gene of the present invention is reduced, it is effective that the DNA or the gene construct of the present invention is introduced for expression into the bodies of the patients by gene therapy using as vehicles appropriate vectors, such as retrovirus vectors, adenovirus vectors and adenovirus-associated virus vectors according to known techniques. Further, when patients cannot exert normal functions because of the increased expression amount, introduction of antisense may be effective.

The DNA, the antisense DNA of the present invention, or the gene construct thereof may be administered alone, or in combination with an adjuvant to promote uptake using a gene gun or a catheter, such as a hydrogel catheter.

In another example, injection of the polypeptide of the present invention or the like into patients with the above diseases also enables the polypeptide of the present invention or the like to exert its function in the patients.

Furthermore, the antibody of the present invention may be used for quantitatively determining the polypeptide of the present invention in a test liquid by a known method. Specifically, the antibody of the present invention may be used for quantitative determination by a sandwich immunoassay using monoclonal antibodies, detection by tissue staining, and the like, by which, for example, diseases that involve the polypeptide of the present invention or the like may be diagnosed.

For these purposes, an antibody molecule itself may be used, or the antibody molecules F(ab′)₂, Fab′ or Fab fractions may be used. Quantitative determination methods for the polypeptide of the present invention using the antibody of the present invention are not specifically limited. Any measurement method can be used, so far as it involves detecting the amount of antibodies, antigens or antibody-antigen complexes corresponding to the amount of antigens (for example, protein amount) in a test liquid by chemical or physical means, and calculating with a calibration curve which has been prepared using a standardized solution containing a known amount of antigens. For example, nephrometry, competitive assay, immunometric assay and sandwich assay are preferably used. Examples of a labeling agent that may be used in a measurement method using a labeling substance include a substance known in the art, such as radioisotopes, enzymes, fluorescent materials and light-emitting materials.

Details about the general technical procedures concerning these measurement and detection methods may be referred to in a review, reference book or the like, such as Radioimmunoassay 2 edited by Hiroshi IRIE, (Kodansha, issued in 1979); Enzyme Immunoassay edited by Eiji ISHIKAWA et al., (3^{rd} edition; Igakushoin, issued in 1987); and Methods in Enzymology (issued by Academic Press), vol. 70, “Immunochemical Techniques (Part A)”, vol. 73, “Immunochemical Techniques (Part B)”, vol. 74, “Immunochemical Techniques (Part C)”, vol. 84, “Immunochemical Techniques (Part D: Selected Immunoassays)”, vol. 92, “Immunochemical Techniques (Part E: Monoclonal Antibodies and General Immunoassay Methods)”, and vol. 121, Immunochemical Techniques (Part I: Hybridoma Technology and Monoclonal Antibodies)”.

Moreover, the DNA chip prepared by arraying the DNA of the present invention is useful in detecting mutations and polymorphism of the DNA of the present invention, and monitoring the DNA dynamics. Regarding DNA array, which is a type of DNA chip, see “DNA microarray and Current PCR method” (a supplementary volume of Cell Technology, Genome Science Series 1, under the editorship of Masaaki MURAMATSU and Hiroyuki NABA, 1^{st} edition, issued on Mar. 16, 2000) and the like.

Further, the polypeptide chip prepared by arraying the polypeptide of the present invention is a strong tool for functional analysis on the expression, interaction and posttranslational modification of the polypeptides of the present invention, and for identification and purification of proteins.

The antibody chip prepared by arraying antibodies against the polypeptides of the present invention is very useful in analyzing the correlation between the polypeptides of the present invention and diseases, disorders, or other physiological phenomena.

Methods and materials for preparing those chips are well known for those skilled in the art.

Furthermore, the polypeptides of the present invention or the like are useful as reagents for screening compounds which interact specifically with these substances. More specifically, the present invention provides a method for screening compounds specifically interact with the polypeptide of the present invention, a partial peptide thereof or salts thereof by using these substances or the salts thereof; and provides the screening kit therefor.

Compounds or salts thereof that are identified by using the screening method or the screening kit of the present invention interact with the polypeptide of the present invention or the like so as to, for example, regulate, inhibit, promote or antagonize the biological activity of the polypeptide of the present invention or the like. The compound or the salt thereof may directly act on the activity of the polypeptide of the present invention or the like, or indirectly act on the activity of the polypeptide of the present invention or the like by acting on the expression of the polypeptide of the present invention or the like. An example of the salt of the compound that is used herein is a pharmaceutically acceptable salt. Specific examples of such salts include a salt formed with inorganic base, a salt formed with organic base, a salt formed with inorganic acid, a salt formed with organic acid, and a salt formed with basic or acidic amino acid. Compounds that inhibit the biological activity of the polypeptide of the present invention or the like may also be used as pharmaceutical preparations, such as therapeutic agents and preventive agents for each of the above-mentioned diseases.

The abbreviation for a base and amino acid is shown in the present specification in accordance with IUPAC-IUB Commision on Biochemical Nomencalture or conventional methods, and an optical isomer of the amino acid, if any, means its L-isomer unless otherwise noted.

### EXAMPLES

The present invention will by further explained by the following examples, which do not limit the scope of the present invention. The genetic procedures in the examples are done in accordance with those described in Current protocols in molecular biology (edited by Frederick M. Ausubel et al., 1987).

(1) Construction of cDNA Library Derived from Human Adult Whole Brain and Human Fetal Whole Brain

A double-stranded cDNA was synthesized by SuperScriptII reverse transcriptase kit (Invitrogen Co.) with the use of an oligonucleotide having NotI site (GACTAGTTCTAGATCGCGAGCGGCCGCCC(T)₁₅) (Invitrogen Co.) as a primer, and mRNA derived from human adult whole brain and human fetal whole brain (Clontech Co.) as a template. An adapter having SalI site (Invitrogen Co.) was ligated with the resulting cDNAs. After digestion with NotI, the cDNAs were subjected to electrophoresis on a low-melting agarose of 1% to purifiy cDNA fragments with 3 kb or more.

The thus purified cDNA fragments were ligated with pBluescript II SK+plasmid treated with SalI-NotI restriction enzymes. The resulting recombinant plasmids were introduced into E. coli DH10B strain (Invitrogen Co.) by an electroporation method.

(2) Screening

The terminal nucleotide sequences of randomly selected clones were determined, and the homology search was done on nr database (all GenBank+EMBL+DDBJ+PDB sequences, but no EST, STS, GSS or phase 0.1 or 2 HTGS seqeunces) with the use of the resulting sequences as a query in accordance with homology search program BLASTN2.2.1 (Altshul, Stephen F., Thomas L. Madden, Alejandro A., Schaffer, Jinghui Zhang, Zheng Zhang, Webb Miller, and David J. Lipman (1997), “Gapped BLAST and PSI-BLAST: a new generation of protein search programs.”, Nucleic acids Res. 25:3389-3402). As a result, the terminal sequences of 3′- and 5′-ends of a gene having no homologous gene, i.e., a novel gene were aligned with human genomic sequence (ftp://ncbi.nlm.nih.gov/genomes/H sapiens/) with the use of homology search program BLASTN2.2.1.

Genes were picked up from a genome region inserted between them by the use of Genscan program (computer software for predicting a gene from genome sequences) (Burge, C. and Karlin, S. 1987, Prediction of complete gene structures in human genomic DNA, J. Mol. Biol., 268, 78-94). Homology search was done on mergedb, which had been prepared by combining human cDNA sequences determined by KAZUSA DNA Institute and Homo sapiens database of GenBank (except EST and genome) without overlapping data, with the use of the selected genes as a query in accordance with homology search program BLASTN2.2.1. When a novel long-ORF gene (with 1,200 bp or more of cds according to the prediction by Genscan) was found, the full-length sequences of its 5′- and 3′-ends were determined.

Determination of the nucleotide sequence was carried out by means of a DNA sequencer (ABI PRISM377) and a reaction kit manufactured by PE Applied Bio System Co. Most of the sequences were determined by a diterminator method on shotgun clones, and parts of them were determined by a primer-walking method with the use of oligonucleotides that were synthesized based on the thus determined nucleotide sequences.

The novel DNAs or genes were screened in the above ways. As a result, a clone fj03204 was found.

(3) Homology Search of the Present DNA

The homology search of the whole nucleotide sequence thus determined was done on the known nr data in accordance with homology search program BLASTP 2.2.1 (Altshul, Stephen F., Thomas L. Madden, Alejandro A., Schaffer, Jinghui Zhang, Zheng Zhang, Webb Miller, and David J. Lipman (1997), “Gapped BLAST and PSI-BLAST: a new generation of protein search programs.”, Nucleic acids Res. 25:3389-3402). As a result, it is revealed that the present DNA has homology to a gene shown in Table 1. Table 1 shows information about the gene (homologous gene) such as its name, data base ID, species, length of protein and references.

TABLE 1

| Homologous | | Length of | | |
|---|---|---|---|---|
| gene | Data base ID | Protein | Species | Reference |
| Quiescin Q6 | gi \| 13325075 | 747 | Human | Genomics(1998), |
| | | | | 54 (3); 460-8 |
| Quiescin Q6 | gi \| 16758172 | 570 | Rat | J.B.C.(2001), |
| | | | | 276(17); 13830-7 |
| Quiescin Q6 | gi \| 12963609 | 568 | Mouse | Genome Res. 2000, |
| | | | | 10(10); 1617-30 |

Table 2 summarizes a variety of data concerning homology between the DNA or the genes of the present invention and each homologous gene listed in Table 1.

The meaning of each item in Table 2 is as follows:

“Score”: the higher this value is, the higher credibility is;

“E-value”: the closer this value comes to “0”, the higher credibility become;

“Homology”: the percentage of identical amino acids in the homologous region; and

“Homology region percentage (%)”: the percentage of the homologous region in the homologous gene.

TABLE 2

| | | | | | Homology Value | | | |
|---|---|---|---|---|---|---|---|---|
| Homologous region | | | | | | | | Percentage of |
| Clone | | Homologous gene | | | | | | the homology |
| from | to | Species | from | to | Score | E-value | Homology | region |
| 34 | 629 | Human | 12 | 613 | 464 | e−135 | 41%(252/611) | 81% |
| 23 | 583 | Rat | 6 | 559 | 474 | e−132 | 44%(246/565) | 97% |
| 23 | 588 | Mouse | 6 | 565 | 483 | e−1129 | 45%(257/572) | 99% |

(4) Search of Domains

Using as queries the amino acid sequence encoded by DNAs contained in the clones, functional domains were searched with a search tool contained in Pfam 7.6 (Pfam HMM ver. 2.1 Search (HMMPFAM), Sonnhammer, E. L. L., Eddy, S. R., Birney, E., Bateman, A., and Durbin, R. (1998) “Pfam: multiple sequence alignments and HMM-profiles of protein domains”, Nucleic Acids Res. 26:320-322).

Further, transmembrane domains were searched with a prediction program for membrane proteins, the SOSUI system (ver. 1.0/10, March, 1996) (Takatsugu Hirokawa, Seah Boon-Chieng and Shigeki Mitaku, SOSUI: Classification and Secondary Structure Prediction System for Membrane Proteins), Bioinformatics (formerly CABIOS) 1998 May; 14(4): 378-379).

Table 3 shows the detected functional domains and transmembrane domains for each clone.

The meaning of each item in Table 3 is as follows:

Functional domain: a domain detected by Pfam or SOSUI

Starting point (From): an amino acid position as a starting point of a functional domain

End point (To): an amino acid position as an end point of a functional domain

Score (Pfam only): the higher the value, the higher the reliability

Exp (Pfam only): the closer the value to 0, the higher the reliability

TABLE 3

| fj03204 | | | | | Human Quiescin | | | | |
|---|---|---|---|---|---|---|---|---|---|
| Functional | | | | | Functional | | | | |
| domain | From | To | Score | Exp | domain | From | To | Score | Exp |
| sosui | 29 | 51 | 28.7 | 6.1e−06 | Thioredoxin | 39 | 155 | 52.5 | 9e−12 |
| Thioredoxin | 60 | 177 | | | | | | | |
| sosui | 654 | 676 | | | | | | | |
| Rat Quiescin | | | | | Mouse Quiescin | | | | |
| Functional | | | | | Functional | | | | |
| domain | From | To | Score | Exp | domain | From | To | Score | Exp |
| sosui | 12 | 34 | 49.0 | 1e−10 | sosui | 11 | 33 | 45.8 | 9.9e−10 |
| Thioredoxin | 42 | 162 | | | Thioredoxin | 42 | 162 | | |
| sosui | 63 | 85 | | | sosui | 63 | 85 | | |

(5) Expression Site

Expressions in the tissues and the sites of the brain were examined by RT-PCR ELISA (Nagase, T., Ishikawa, K., Suyama, M., Kikuno, R., Miyajima, N., Tanaka, A., Kotani, H., Nomura, N. and Ohara, O. Prediction of the coding sequences of unidentified human genes. XI. The complete sequences of 100 new cDNA clones from brain which code for large proteins in vitro. DNA Res. 1998 Oct. 30; 5(5): 277-86). Table 4 shows the result.

The expression amount (unit (fg) per ng of poly(A)+ RNA) is represented by + for less than 0.1; ++ for more than 0.1, less than 100; and +++ for more than 100.

Table 5 shows the complete notation of each tissue and site of the brain.

TABLE 4

| | Adult | | | | | | | | | | | |
|---|---|---|---|---|---|---|---|---|---|---|---|---|
| | | | | | | | | | | | Site of | |
| Clone | Tissue | | | | | | | | | | the brain | |
| name | He | Br | Lu | Li | Sm | Ki | Pa | Sp | Te | Ov | Am | Co |
| fj03204 | +++ | +++ | +++ | +++ | +++ | +++ | +++ | +++ | +++ | +++ | +++ | +++ |

| | | |
|---|---|---|
| | Adult | Embryo |
| Clone | Site of the brain | Tissue |

| name | Ov | Co | Ce | Ca | Hi | Ni | Nu | Th | Sp | Li | Br |
|---|---|---|---|---|---|---|---|---|---|---|---|
| fj03204 | +++ | +++ | +++ | +++ | ++ | +++ | +++ | +++ | +++ | +++ | +++ |

TABLE 5

| | Abbreviated notation | Complete notation |
|---|---|---|
| Tissue | Br | Brain |
| | He | Heart |
| | Ki | Kidney |
| | Li | Liver |
| | Lu | Lung |
| | Ov | Ovary |
| | Pa | Pancreas |
| | Sm | Skeletal muscle |
| | Sp | Spleen |
| | Te | Testis |
| Site of the brain | Am | Amygdala |
| | Ca | Caudate nucleus |
| | Ce | Cerebellum |
| | Co | Corpus callosum |
| | Hi | Hippocampus |
| | Ni | Substantia nigra |
| | Nu | Subthalamic nucleus |
| | Th | Thalamus |
| | Sp | Spinal cord |

(6) Location on Chromosome

Using the DNA base sequence of the clones as queries, the above-mentioned analysis program BLASTN 2.2.1 (“Gapped BLAST and PSI-BLAST: a new generation of protein database search programs”) was run on the human genome sequences in the library of known sequences, Genbank release 122 and 123. Additionally, using the homology search program BLASTN 2.2.1, the DNA sequence of the clone was aligned with the library of clones encoding human genome (ftp://ncbi.nlm.nih.gov/genomes/H_sapiens/). As a result, it has been revealed that the present DNA is located on chromosome 2 (2q21).

(7) Expression of the Protein Encoded by the Present Gene

A gene product was expressed from the cDNA clone fj03204 with the use of the transcription and translation system in vitro (Promega Co., TNT T7 Quick Coupled Transcription/Translation System cat.no.L1107).

The product incorporated with ³⁵S-labeled methionine was subjected to SDS-PAGE (12.5%). After drying of a gel, autoradiography was done with the use of BAS2000 (Fuji film) system to detect the gene product of the clone fj03204. As a result, a band, which was presumed to be a transcription/translation product of the clone fj03204, was observed at a point corresponding to a marker with 77 kDa.

As a molecular weight of the protein encoded by the fj03204 consisting of 698 amino acids from a first methionine is presumed to be about 77,350 Da, the presumed molecular weight was coincided well with the above result.

(8) Function of the Present Gene

The data concerning homology, information about the homologous genes, domains, expression sites and location on chromosome have revealed the followings:

The searches of homology and functional domains clarified that the present DNA or gene has about 40% homology with Quiescin Q6 Gene family, and has a thioredoxin domain at its N-terminus. Further, as seen from alignment with the amino acid sequences of Quiescin Q6 Gene (QSCN 6) disclosed in Non-patent documents 1 and 2, the present DNA or gene has ERV1 of yeast at its C-terminus, i.e., amino acids 405-539 in SEQ ID No.1. These facts suggest that the present DNA or gene belongs to Quiescn Q6 Gene family (QSCN 6) as disclosed in Non-patent documents 1 and 2.

As described in Non-patent documents 1 and 2, ERV1 gene is essential for oxidative phosphorylation and asexual reproduction of yeast (Lisowsky, T. (1992), Mol. Gen. Genet., 232:58-64), and play an important role in maintenance and cell division cycle of yeast mitochondria genome (Lisowsky, T. (1994), Curr. Genet., 26:15-20).

QSCN6 is lowly expressed in a fibroblast that is actively growing, and highly expressed in that in quiescent phase. While the expression of mRNA of QSCN6 is strongly induced at the time when the fibroblast begins to shift from growth phase into quiescent phase, it is strongly inhibited in a transformed fibroblast (Coppock D. L. et al., (1993) Cell Growth Differ., 4:483-493).

Some of the other genes belonging to the above group are a component of extracellular matrix (ECM), which plays not only a structural role in a cell but also a functional role such as suppression of tumor or regulation of growth.

From the above information, it seems that QSCN6 may possibly play a role in cell adhesion. It may further function during the entering process of a normal cell into an irreversible quiescent phase. It is also suggested that the inhibition of this gene may play a part in canceration (malignant alternation). Further it seems that QSCN6 may play a role in control of cell growth and redox state.

Accordingly, those skilled in the art may reasonably presume that the present DNA or gene has function that is deeply related to diseases involved aging or cancer.

As seen form the base sequence of clone fj03204 represented by SEQ ID No.1, the surrounding base sequence (AACATGG) of its translation initiation codon coincides well with Kozak consensus sequence (ACCATGG). Furthermore, while QSCN6 protein exists in matrix of yeast mitochondria as described in Non-patent document 2, a signal sequence necessary for secretion (the region detected by Sosui program) is found at the N-terminus of the clone fj03204. This fact means that the clone fj03204 encodes the gene with a full length.

### INDUSTRIAL APPLICABILITY

Cell growth is a dynamic process that is determined by the ratio among a fraction of cells in growth phase, a fraction of cells in quiescent phase, and a fraction of dead cells. The regulation of transition from the growth phase to the quiescent phase is an important step in the whole regulation of a growth process. The inhibition of a normal transition into the quiescent phase is characteristic in cancer and other proliferative diseases.

It is expected therefore that the present invention contributes to diagnosis and treatment of the diseases involved aging or cancer.

Further, a single nucleotide polymorphism, SNP, which is a change in one base (nucleotide) among individuals in the DNA or the gene of the present invention, can be found by performing PCR using synthetic DNA primers prepared based on the nucleotide sequence of the DNA or the gene of the present invention or a part thereof, and using chromosome DNA extracted from human blood or tissue so as to determine the nucleotide sequence of the product. Therefore, individual constitution or the like can be predicted, which enables the development of a pharmaceutical preparation suitable for each individual.

Further, when ortholog (homolog, counterpart) genes for the DNA or the gene of the present invention in model organisms, such as mice, are isolated with cross hybridization, for example, these genes are knocked out to produce human disease model animals, so that the causative genes which cause human diseases can be searched and identified.

Novel DNAs or genes obtained by the present invention are assembled on a so-called DNA chip, and then probes prepared using blood or tissue derived from cancer patients or patients with diseases that relate to the brain, such as mental disease, or as a control using blood or tissue from healthy individuals are hybridized to the chip, so that the chip can be applied to diagnosis and treatment for the diseases.

Moreover, antibody chip, on which the antibodies against the polypeptides of the present invention are thoroughly prepared and arrayed, can be applied to diagnosis, treatment of diseases and the like through proteome analysis, such as detection of a difference in expression amount of a protein between a patient and a healthy individual.

Furthermore, the DNAs or the gene constructs of the present invention can be used as an active ingredient of vaccine.

The present application asserts priority based on the specification of Japanese Patent Application No. 2002-205915 and includes by reference all of the contents as disclosed in the specification.

1. A DNA comprising a base sequence encoding the following polypeptide (a) or (b):
(a) a polypeptide consisting of an amino acid sequence which is identical or substantially identical with the amino acid sequence represented by SEQ ID No.1,
(b) a polypeptide consisting of an amino acid sequence represented by SEQ ID No.1 in which part of amino acids are deleted, substituted or added, and having substantially the same biological activity as the function of the polypeptide (a).

2. A DNA hybridizing with a DNA having a base sequence complementary to the DNA of claim 1 under stringent conditions, and having substantially the same biological activity as the function of the above polypeptide (a) of claim 1.

3. A gene construct comprising the DNA of claim 1 or 2.

4. A polypeptide (a) or (b):
(a) a polypeptide consisting of an amino acid sequence which is identical or substantially identical with the amino acid sequence represented by SEQ ID No.1,
(b) a polypeptide consisting of an amino acid sequence represented by SEQ ID No.1 in which part of amino acids are deleted, substituted or added, and having substantially the same biological activity as the function of the polypeptide (a).

5. A recombinant polypeptide encoded by the gene construct of claim 3.

6. An antibody against the polypeptide of claim 4 or 5.

7. A DNA tip on which the DNAs of claim 1 or 2 are arrayed.

8. A polypeptide tip on which the polypeptides of claim 4 or 5 are arrayed.

9. An antibody tip on which the antibodies of claim 6 are arrayed.

10. An antisense oligonucleotide to the DNA of claim 1 or 2.
